(19) United States
(12) Patent Application Publication
(10) Pub. No.: US 2003/0171655 A1
(43) Pub. Date: Sep. 11, 2003

(54) Combination otoscope

(76) Inventors: Richard W. Newman, Auburn, NY (US); Dominick Danna, Syracuse, NY (US)

Correspondence Address: WALL MARJAMA & BILINSKI, 101 SOUTH SALINA STREET, SUITE 400, SYRACUSE, NY, 13202, US

(21) Appl. No.: 10/094,121

(22) Filed: Mar. 8, 2002

(51) Int. Cl. A61B 1/267

(52) U.S. Cl. 600/200

(57) ABSTRACT

An otoscopic instrument permits visual inspection of the ear canal and includes at least one chemical sensing element for detecting a fluid indicative of a disease as well as at least one of an infrared thermometer, and a mechanism for determining pressure variations and the presence of fluid in the ear for providing a comprehensive examination of a patient. The instrument can also be adapted for use with other body cavities such as the throat and nose, among others.

### FIELD OF THE INVENTION

The invention relates to the field of medical instruments, and more particularly to a medical diagnostic instrument capable of providing a complete examination of the ear and other similar medical target areas.

### BACKGROUND OF THE INVENTION

Otoscopes have long been a staple device in the physician's office as a tool for visually examining the ear. A typical otoscope, such as those manufactured by Heine Inc. and Welch Allyn, Inc., among others, includes a hand-grippable battery handle having an instrument head mounted to the top of the handle. A conical speculum portion at a distal end of the instrument head permits insertion thereof a predetermined distance into the ear canal of a patient. An image is seen by the user through means of a magnifying eyepiece located on the rear or proximal side of the instrument, with the ear being illuminated by means of an interior lamp or a lamp tethered to a bundle of optical fibers located in the instrument head to facilitate viewing.

Other otoscopic instrument versions have since been developed which include a video camera that is attached to the eyepiece portion of the instrument head. An optical lens system, such as a relay lens assembly or a rod lens assembly, transmits the image directly to the camera. More recent versions employ a miniature imager element, such as a CCD, which is distally or otherwise positioned within the instrument head, an example being described in commonly assigned U.S. Pat. No. 5,919,130, the entire contents of which are herein incorporated by reference.

Pneumatic otoscopy employs a pneumatic bulb which is fluidly connected to a sealed interior of the insertion portion of the otoscope so as to create alternately a pressure and a vacuum within the ear canal. This technique, referred to as insufflation, allows vibration of the tympanic membrane.

There is an overall compelling need in the field to be able to provide a more accurate and complete examination/diagnosis of the ear. That is, an accurate examination of the tympanic membrane and the ear canal can be invaluable as to the correct prescribing of appropriate antibiotics in children as well as the diagnosis of otitis (inflammation of the ear) and other ear-specific maladies.

For example, an ear afflicted with chronic otitis typically has a “normal” temperature, as related to body temperature, with a clear fluid being present behind the tympanic membrane. In contrast, acute otitis produces an ear of raised temperature with an opaque fluid with exudate being present behind the tympanic membrane. In the acute case, it is often difficult to see through the tympanic membrane with visible light, therefore it is difficult to know the extent of the fluid which is present in the middle ear. It is also difficult to know if the infection is viral or bacterial in nature.

Certain technologies, such as tympanometry and acoustic reflectance, have emerged which facilitate the diagnosis of otitis and other ear-related maladies. However and to date, none of these technologies have been combined, for example, with visual otoscopy. Furthermore, all of these emerging technologies predominantly examine only a portion (e.g., outer, middle) of the ear. To date, a single instrument has not been developed which allows the entirety of the ear to be thoroughly examined.

### SUMMARY OF THE INVENTION

It is a primary object of the present invention to improve the field of medical diagnostic instruments.

It is another primary object of the present invention to provide a single medical diagnostic instrument that allows several different and separate diagnostic procedures to be performed to provide a more comprehensive and reliable patient examination.

It is yet another primary object of the present invention to provide a medical diagnostic instrument which is capable of examining the inner and middle ear, as well as the outer ear, so as to more reliably provide a more thorough and complete diagnosis. More preferably, it is an object to provide an ear diagnostic instrument which can detect numerous physical parameters associated with, for example, otitis, hearing, body temperature, respiration, and pulse.

Therefore and according to a preferred aspect of the invention, there is provided a medical diagnostic instrument comprising at least one chemical sensor for determining the presence of a fluid indicative of a disease, and viewing means for viewing a medical target of interest.

Preferably, the instrument includes an insertion portion capable of being inserted to a predetermined distance into the ear canal of a patient, in which the at least one chemical sensor is disposed within the insertion portion.

The viewing means can include at least one of a viewing optic aligned with the insertion portion or with an imaging sensor which is disposed within the insertion portion or otherwise attached to the instrument.

The herein described instrument can further include temperature measuring means for determining the body temperature of a patient. According to a preferred aspect, the temperature measuring means can include at least one infrared (IR) sensor or sensor array capable of providing a thermal image of a medical target, which can provide an indication of body temperature. Moreover, core body temperature can also be determined through this type of inspection.

In addition, the temperature measuring means can further determine the pulse rate of the patient.

The above diagnostic instrument can further include means for detecting the presence of fluid in the middle ear, and/or pressure measuring means for measuring pressure variations in the middle ear. The fluid detecting means can include, for example, at least one of pneumatic otoscopy, oto-reflectance, acoustic reflectance, impedance reflectance, and tympanometry means for detecting the presence of fluid in the middle ear.

In addition, the above instrument can include a microphone capable of picking up inspiration sounds in order to determine the respiration rate of a patient.

The instrument can further include spectroscopic means for determining the presence of pathogens, the spectroscopic means including a light source capable of emitting predetermined wavelengths of light and detecting means for detecting predetermined wavelengths of light emitted within the ear.

Still further, the herein described instrument also includes means for determining hearing loss in the inner ear in which these means includes at least one of otoreflectance and otoacoustic emission means.

The herein described instrument though primarily designed to examine the ear can also be used at least in part, to examine other body cavities such as the nose and throat, among others. For example, the instrument can be used to determine the presence of strep throat both within the ear and the throat of a patient using the chemical sensing element(s) and/or temperature measuring means.

According to another preferred aspect of the invention, there is provided an otological instrument comprising oto-reflectance means for determining at least one of detecting the presence of fluid in the middle ear and hearing in the inner ear; and at least one chemical sensor for determining the presence of a disease.

According to yet another preferred aspect of the invention, there is provided an instrument for determining the condition of a medical target, said instrument comprising means for viewing the medical target, and temperature measuring means for measuring body temperature of a patient, said temperature measuring means including at least one sensor capable of producing an output signal which is indicative of temperature and processing electronics for processing output signals from said at least one sensor.

According to still another preferred aspect of the invention, there is provided an instrument for determining the condition of an ear, said instrument comprising means for viewing the ear interior; and means for measuring pressure variations within the ear.

According to yet another preferred aspect of the present invention, there is provided a diagnostic instrument comprising means for viewing a medical target, and sensing means for detecting the presence of pathogen at the medical target.

According to still another preferred variation of the present invention, there is provided an instrument for determining the condition of the ear, said instrument comprising temperature measuring means for determining the body temperature of a patient, said temperature measuring means including at least one miniature infrared sensor capable of providing a thermal image of the interior of the ear, and pressure measuring means for measuring pressure variations within the middle ear.

According to still another preferred variation of the present invention, there is provided an instrument for determining the condition of a medical target, said instrument comprising temperature measuring means for determining the body temperature of a patient, said temperature measuring means including an array of sensors capable of providing a thermal image of the medical target, such as the interior of the ear and sensing means for sensing the presence of pathogens indicative of an ear or other medical condition.

According to yet another preferred variation of the present invention, there is provided a medical instrument system capable of comprehensively examining the ear, said system comprising a housing having an insertion portion sized for fitting a predetermined distance within the ear canal of a patient, means for examining the outer ear means for examining the middle ear, and means for examining the inner ear of a patient, each of said examining means being at least partially contained within said housing.

According to yet another preferred variation of the present invention, there is provided an otological instrument comprising pressure determining means for determining pressure variations in the middle ear, and means for sensing the presence of pathogens in the ear.

Preferably, the instrument is an otoscope which allows the ear canal, tympanic membrane, middle ear and ossicles to be viewed, either visually or using a miniature video camera. This instrument includes a handle having an attached instrument head, the instrument head having a distal speculum portion which can be fitted a predetermined distance into the ear canal of a patient. The instrument is preferably hand-held, the instrument head being either integrally or releasably attached to the handle.

The instrument permits visual inspection through use of an eyepiece or provides a video signal of a target of interest which can be displayed.

According to a preferred embodiment, the instrument includes a thermal sensor array which permits thermal imaging of the ear canal. The array includes a plurality of miniature infrared sensors which emit and detect temperature differences upon a scanned or interrogated area. Using such an array in conjunction with an ear diagnosis device permits the tympanic membrane to be discriminated from other portions of the ear which are prone to transient thermal conditions resulting in a more accurate reading of core body temperature as well as the determination of any localized “hot” spots which could be indicative of infection(s) or inflammation(s).

Visual inspection is permitted using either the physician's eye or alternately by means of a contained or attached video camera. By combining visual capability with, for example, pneumatic otoscopy or tympanometry, the physician or care giver can see redness, bulging of the TM, lack of motion of the TM, and other visually discernible features.

Preferably, the ear or other suitable medical target can further be illuminated with IR radiation with the reflected light being sensed by a thermal sensing array contained within the device. The IR radiation would pass through the TM and be reflected depending on the nature of air, fluid (opaque or clear) behind the TM, presence of bubbles and the like. As such, the presence of fluid can be detected. Moreover, the type of fluid can be distinguished from the reflected light. According to a preferred version of the device, an array containing miniature chemical sensors capable of detecting vapors contained in a disease process to identify certain pathogens, for example certain viruses and bacteria, which may be present as a result of otitis or other detectable condition. Moreover, a thermal image, spectroscopic image, and/or other image(s) can be superimposed onto a video/optical image to enhance or otherwise improve diagnosis.

Another feature of the present invention is that pressure measuring means can be provided in the instrument to permit measurement of fluid and pressure behind the TM, such as, for example, at least one tympanometric device, an oto-reflectance device, and/or an impedance reflectance device by providing at least one microphone and an acoustic source such as a speaker calibrated with the at least one microphone within the head of the instrument for exposing same to the interior of the ear.

Inclusion of a microphone for the pressure measuring means can further be utilized in the herein described device in order to determine the respiratory rate of a patient who is being examined. Furthermore, the above referred to thermal imaging array can also determine the pulse rate of the patient based on transient changes in temperature gradient. Determination of the temperature gradient can also predict body core temperature should the presence of an abscess or other obstruction be found.

An advantage of the present invention is that a more accurate and complete examination of the ear or other medical target can be performed using a single diagnostic instrument.

Another advantage of the present invention is that acute and chronic otitis can be distinguished and diagnosed, along with other ear-related maladies, using a single instrument.

Yet another advantage provided by the present invention is a more detailed analysis and examination of the patient, allowing antibiotics to be prescribed more judiciously.

Still another advantage is that the instrument can be configured to permit examination of the throat using either an IR thermal array and/or the at least one chemical sensing element for example, to detect the presence of pathogens indicative of certain ear-related maladies or diseases ranging from cancer to strep throat. In the case of the latter, the herein described otoscopic instrument can also be used to interrogate the throat or other medical target following an ear diagnosis.

These and other objects, features, and advantages will be apparent from the following Detailed Description which should be read in conjunction with the accompanying drawings.

### BRIEF DESCRIPTION OF THE DRAWINGS

FIG. 1 is a partial sectional side view of a medical diagnostic instrument in accordance in the known art;

FIG. 2 is a partial sectional side view of a medical diagnostic instrument according to a first embodiment of the present invention;

FIG. 3 is a partial sectional side view of a medical diagnostic instrument according to a second embodiment of the present invention;

FIG. 4 is a partial sectional side view of a medical diagnostic instrument according to a third emdodiment of the present invention;

FIG. 5 is a partial sectional side view of a medical diagnostic instrument according to a fourth embodiment of the present invention; and

FIG. 6 is a partial sectional side view of a medical diagnostic instrument according to a fifth embodiment of the present invention.

### DETAILED DESCRIPTION

The following discussion relates to an otological instrument in accordance with several embodiments. It will be understood that other instrument designs employing the inventive concepts described herein will be readily apparent. That is, the instrument can also be used for examining other medical targets, such as the nose and throat, among others. In addition and throughout the course of the discussion which follows, terms such as “top”, “bottom”, “distal”, “proximal”, “upper”, “lower” and the like are used in order to provide a frame of reference for the accompanying drawings. These terms, however, should not be construed to be limiting of the invention as defined herein by the appended claims. The term “fluid” is used repletely throughout the discussion. As used, this term is intended to encompass liquids, vapors and/or gases. The term “image” is also used frequently and refers to any captured, still, or moving representation.

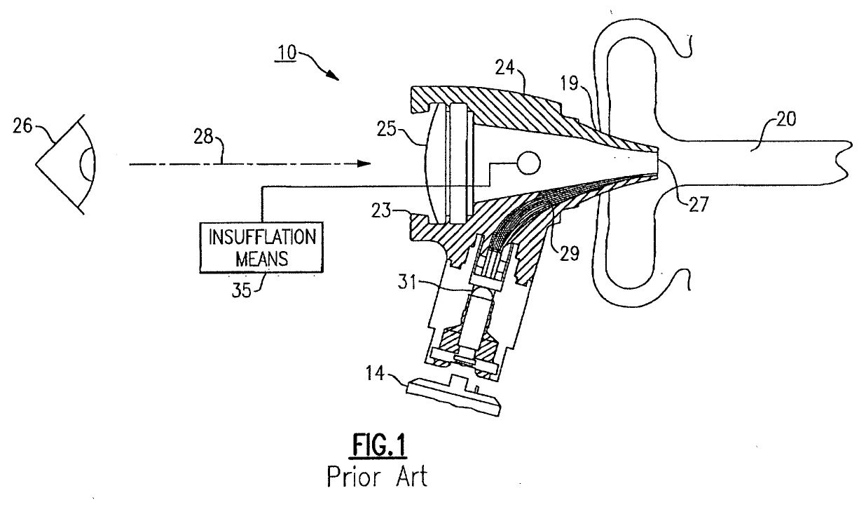

Referring to FIG. 1, there is illustrated a otoscopic instrument 10 in accordance with the known art. The instrument 10 includes a cylindrical hand-grippable handle 14, partially shown, the handle having an interior adequately sized to retain a pair of batteries (not shown) serving as a power source for the instrument. Alternately, the instrument 10 can be configured for attachment to another power source, such as a wall transformer (not shown). An instrument head 24, attached in a known manner to the top of the handle 14, is defined by a substantially frusto-conical distal insertion portion 19 which is sized to be positioned a predetermined distance into the ear canal 20 of a patient. A disposable tip (not shown) can be suitably attached in overlaying fashion onto the exterior of the distal insertion portion 19.

The instrument head 24 is essentially hollow and includes a proximal end 23 having a magnifying eyepiece 25 to permit viewing of a target through a distal tip opening 27. For each of the herein described embodiments, and in order to adequately illuminate the target of interest, a fiber optic bundle 29 transmits light from a light source, such as a miniature halogen lamp 31, that is disposed in the neck of the handle 14. It will be readily apparent, however, that the invention is not intended to be limited to the herein described illumination system and that alternate light sources, such as for example, LEDs, laser diodes, and the like can be substituted for the halogen lamp 31. Visual otoscopy using the above instrument 10 permits the ear canal, middle ear, and ossicles to be readily observed for diagnosis by a physician through the eyepiece 25.

The herein described prior art otoscope 10 further includes means for providing a subjective measure of pressure and fluid in the middle ear, such as an depressible bulb, shown diagrammatically as 35 in FIG. 1, in order to vibrate the tympanic membrane, e.g., that is, to insufflate the patient.

To complete the preceding background, it should be further noted that other known otoscopes can be configured for use, such as described in commonly owned U.S. Pat. No. 5,919,130, the entire contents of which are herein incorporated by reference. The above-referenced otoscope includes a miniature solid state imager, such as a CCD, which is disposed within the distal or insertion portion 19 of the instrument head 24, in lieu of the magnifying eyepiece 25, to provide a videoized image of the target. The above referred to otoscope can also include similar insufflation means (e.g., a pneumatic bulb) in a manner known to those of sufficient skill in the field and for which no further details are required.

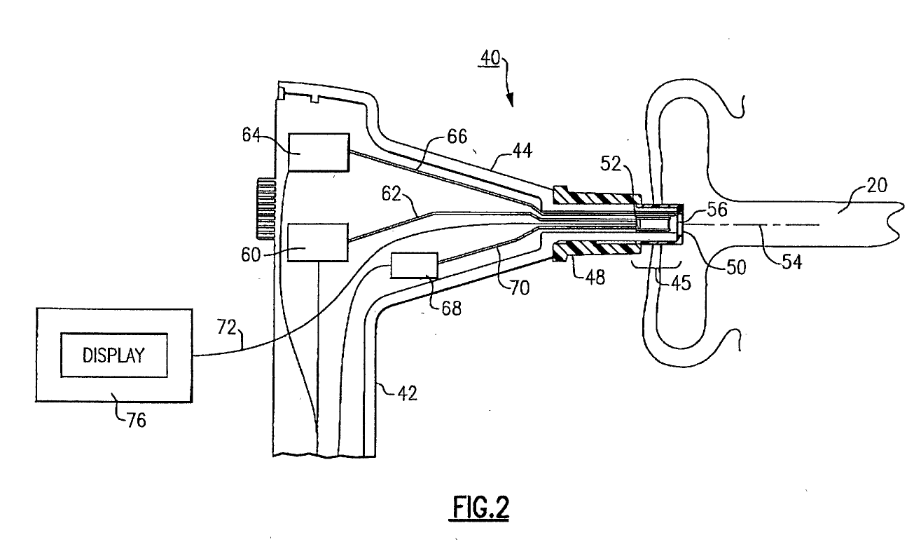

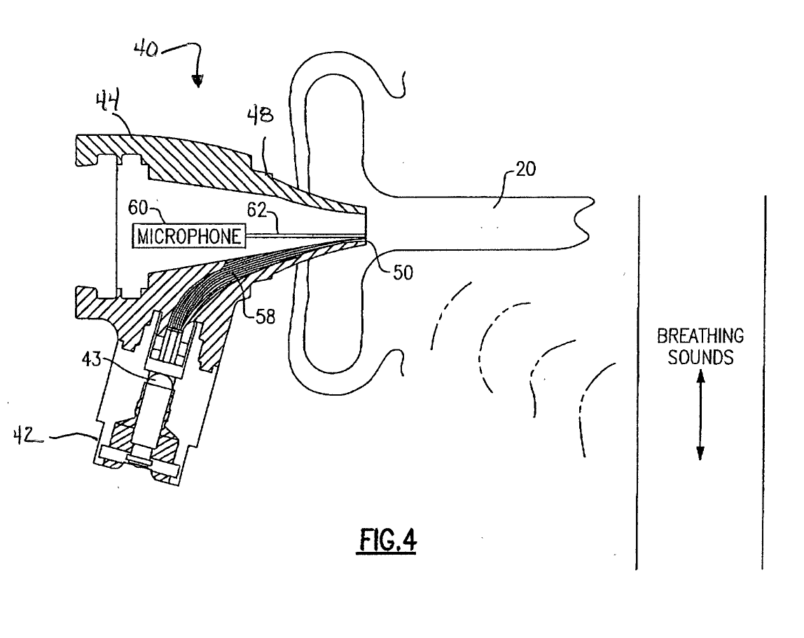

Turning to FIGS. 2 and 4, a medical diagnostic instrument, in this instance, an otological instrument 40 made in accordance with a first embodiment of the present invention is herein described. For purposes of the following discussion, it should be noted that FIGS. 2 and 4 are intended to illustrate instruments having some common characteristics though the detail of each of the FIGS. 2 and 4 are different. For example, FIG. 4 illustrates an otoscopic instrument having only a microphone contained within the instrument head while the otoscope of FIG. 2 contains several components including the microphone. On the other hand, the instrument of FIG. 2 is intended to contain a fiberoptic bundle extending from a miniature light source as shown according to FIG. 4, but which for purposes of clarity is not shown in FIG. 2.

Referring more particularly to FIG. 2, the herein described instrument 40 includes an instrument head 44 having a distal insertion portion 48 having a substantially conical or frusto-conical shape which is configured to permit insertion a predetermined distance into the ear canal 20, FIG. 1, of the patient. The instrument head 44 is similar in construction to the instrument head 24, FIG. 1, described above, including a hollow interior having a distal tip opening 50. In lieu of an eyepiece, a video imaging assembly 45 is disposed within the hollow interior of the instrument head 44. The video imaging assembly 45 comprises a miniature electronic imager 52, such as a CCD, disposed in relation to the distal tip opening 50 of the instrument head 44 along an optical axis 54. A lens cell 56 having at least one objective lens element for focusing incoming light is disposed in relation to an image recording surface of the electronic imager 52 along the optical axis 54. A fiberoptic bundle 58, shown only in FIG. 4, is preferably circumferentially positioned about the exterior of the lens cell 56 and the miniature imager 52, the bundle including light transmitting ends that are disposed in relation to the distal tip opening 50 extending to a light source, such as a halogen lamp 43, also shown only in FIG. 4, contained within the neck 42 of the instrument 40.

In operation, the electrical output signals of the miniature electronic imager 52 are sent along transmission lines 72 to a video display 76 or other peripheral device, the signals either being processed by circuitry in or adjacent the video imaging assembly 45 or alternately by processing circuitry provided within the display 76. The video display 76 can be located remotely or attached to the instrument head 44 or otherwise attached to the instrument 40 and is capable of displaying a various number and assortment of images and image types, as described below. Alternately, a low power miniature CMOS type imager can be used in lieu of the CCD, the CMOS-type imager having discrete processing circuitry located within the imager chip. Such devices are described for example in Fossum et al., U.S. Pat. No. 5,841,126, the entire contents of which are herein incorporated by reference.

According to this embodiment, a number of additional components are disposed within the hollow interior of the instrument head 44 along with the above described video imaging assembly 45, including a microphone 60 and an acoustic source, such as a speaker 64 as well as a miniature pressure pump 68, such as those manufactured by Etymotic Research of ElkGrove Village, Ill. Each of these contained components can either be powered by batteries (not shown) contained either within the handle 42 or alternately the instrument 40 can be powered by an external source such as a wall transformer (not shown).

The contained microphone 60, speaker 64, and miniature pressure pump 68 permit a number of different measurements to be performed. First, these components in combination with the herein described video (visual) assembly permit tympanometric measurements to be performed within the middle ear in order to determine the acoustic admittance/impedance of the middle ear over a single frequency or over a discrete range of frequencies. In general, the miniature pump 68, is a diaphragm or other form of reversible pump capable of creating positive and negative pressures in the ear canal with the ear canal being sealed due to the fit created by the distal insertion portion 48 of the instrument head 44. The above components are preferably connected by known means to the batteries (not shown) contained within the handle 42 of the otoscope instrument 40 or to another suitable power source (not shown). Moreover, the miniature pressure pump 68 produces a range of pressures into the ear canal while acoustic signals are being transmitted by the speaker 64 with a reflected signal being received by the microphone 60, each of which are suitably calibrated in a known manner. Circuit means (not shown) provided within the instrument 40 control the operation of the pump 68, the speaker 64 and microphone 60 and further process the signals received by the microphone in order to derive the acoustic admittance. The above tympanometric components can diagnose, for example, ossicular disarticulation, perforated or scarred tympanic membrane, ossification of the ossicles, and stapedius muscle reflex. Additional details relating to a hand-held tympanometric instrument are described in U.S. Pat. No. 4,688,582, the entire contents of which are herein incorporated by reference.

In addition to tympanometry, other forms of measurements can be made using the acoustic source and microphone of the herein described instrument to provide an objective measure of the pressure and fluid in the middle ear in conjunction without recourse to providing a pneumatic seal with the ear canal and therefore without requiring a pump. As noted above, tympanometry requires a pump to alternately put a static pressure and a vacuum up against the tympanic membrane.

Still referring to FIG. 2, the above microphone 60 and speaker 64 can also be utilized, such as in combination with the video assembly 65, to perform any one of acoustic reflectometry, impedance reflectance, oto-acoustic emissions, or oto-reflectance measurements. Each of the above techniques utilize an acoustic source, such as a speaker, to emit/transmit an acoustic signal(s) to the ear canal which is reflected and detected by the microphone or other form of pressure transducer. Impedance reflectance is similar to tympanometry and measures impedance of the middle ear which is the inverse of admittance and hence can indicate hearing loss and the presence of fluid in the middle ear. Acoustic reflectometry and impedance reflectance each utilize the reflected pressure signal. Oto-reflection is a method which has been advanced since tympanometry. Devices using this diagnostic technique do not necessarily require a pump, therefore this technique is arguably less expensive and faster than tympanometry, especially multi-frequency tympanometry. Oto-reflection allows a determination to be made by means of measuring simultaneously a linear and a nonlinear response to an acoustic stimulii applied by the speaker 64 by examining a power-based reflection function as opposed to pressure. The linear portion of oto-reflection provides an indication of the middle ear while the nonlinear function provides indications relating to the inner ear with each of the linear and nonlinear functions being discernible and therefore identifiable. A more detailed discussion of otoacoustic emission and oto-reflectance is provided in U.S. Pat. Nos. 5,594,174 and 5,792,072, each to Keefe et al, the entire contents of which are herein incorporated by reference. Using this technique, both the middle ear and the inner ear can be diagnosed with a single ear probe simultaneously using the microphone 60 and speaker 64 in combination with the above video assembly.

Still further and referring to FIG. 4, the simple inclusion of a microphone 60 within the instrument head 44 can provide additional data which can be used to detect the sounds of breathing (respiration sounds) in the body of a patient. Since inspiration sounds are typically louder than respiration sounds, it is possible to determine to distinguish between inspiration and respiration, and therefore be able to determine respiratory rate.

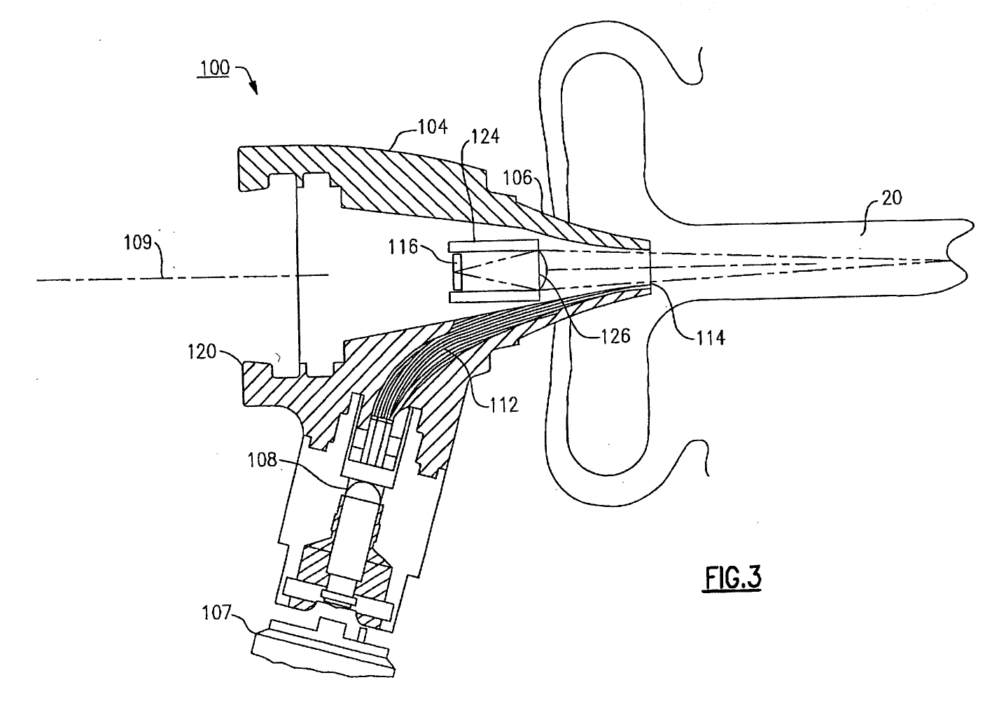

Referring to FIG. 3, an otoscopic instrument 100 in accordance with another embodiment of the invention includes an instrument head 104 having a distal insertion portion 106, the instrument head being attached to the top of an instrument handle 107. A light source 108, such as a miniature halogen bulb, is contained within the neck of the instrument 100 and an optical fiber bundle 112 coupled to the light source provides illumination to a target through a distal tip opening 114. A plurality of miniature infrared (IR) sensors, such as those manufactured by TI/Raytheon, are arranged in a one or two-dimensional array 116 that is provided within the instrument 100 along an optical axis 109 in alignment with the distal tip opening 114 and focusing lens system 126, each of the sensors of the array being coupled to processing means and a display, such as an LED, which is capable of providing a composite thermal image of the target in combination with other functions of the herein described instrument. The processing means converts the electrical signals obtained by each of the sensors into a form suitable for outputting. For example, a thermal profile can be displayed using false colors to identify regions of like temperature within the sensed target area. Alternately, the processing means can simply indicate or predict the highest temperature(s) which have been sensed by the thermal array 116.

Infrared radiation sensors can be employed to determine or estimate the core body temperature of the patient. It is known that the tympanic membrane receives its blood flow from the hypothalamus which in effect is the human thermostat and which is indicative of core temperature. Therefore, detection of the blood vessels within the tympanic membrane by the thermal sensing array 114 provides a measure of the temperature of the hypothalamus. This recognition signifies that greatest accuracy of body temperature can be realized, without requiring that the entire tympanic membrane be analyzed. A thermal array disposed within the instrument or the speculum portion of the instrument can readily be used for making a reasonable determination of body temperature. The above sensing technique can simultaneously be used to measure the heart pulse rate of the patient by looking at a transient thermal profile for surges in heat during each pulse. Additional details relating to the structure and operation of the above thermal sensing array, including multiple display types and detection of pulse rate, for an otoscopic ear thermometer having at least one IR sensor are provided in copending U.S. Ser. No. 09/825,478, the entire contents of which are herein incorporated by reference.

Though the instrument of FIG. 2 is effective; that is, each of pneumatic otoscopy, tympanometry, oto-reflection, and acoustic and impedance reflectance are each capable of determining whether fluid is present behind the tympanic membrane, none of the above techniques are capable of determining the presence of pathogens (e.g., bacteria or virus) in the fluid. Similarly, there is no manner of determining whether or not the bacteria is alive or dead. If live bacteria is present, then it is essential to the treatment of the patient to know the type of bacteria in order to affect proper treatment.

It is known that spectroscopy techniques can be used to observe a combination of fluorescence, reflectance, absorption, and/or scattering of light emitted by a medical light source having at least one predetermined wavelength to observe at least one bacteria or virus in either an in vivo or in vitro biological tissue. Details relating to the detection and examination of such tissues are provided, for example, in Bacteria Identification of Otitis Media with Fluorescence Spectroscopy, Lasers in Surgery and Medicine 14:155-163 (1994) by Sorrell, Tribble, Reinish, Werkhaven, and Ossoff to the entire contents of which are herein incorporated by reference.

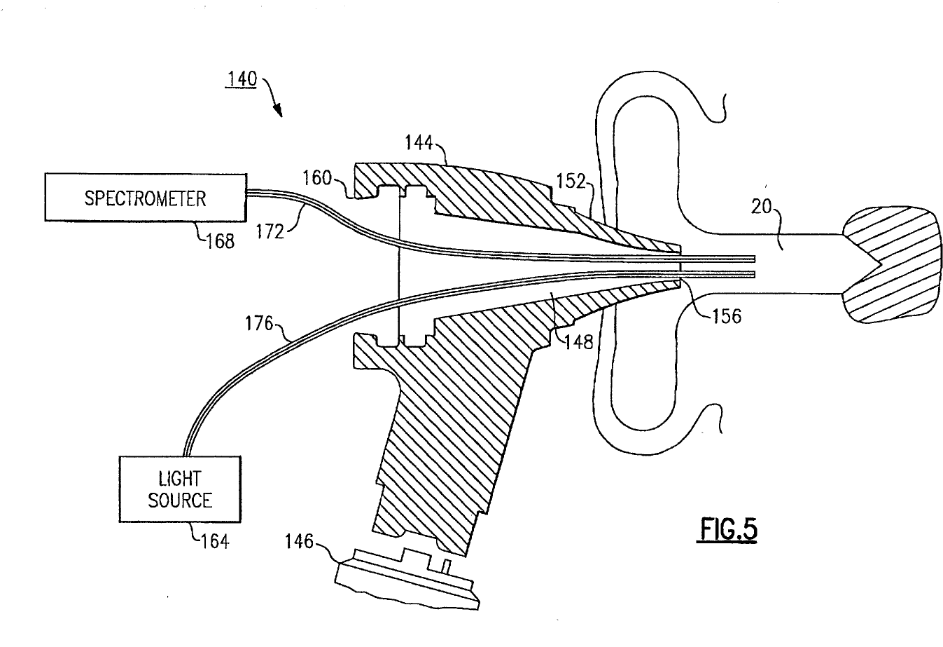

Referring to FIG. 5, an otoscopic instrument 140 is herein described which includes an instrument head 144 attached as previously noted to an instrument handle 146. The instrument head 144 is essentially hollow and includes a frusto-conical distal insertion portion 152 and a proximal end 160 which permits optical viewing (e.g., visual otoscopy) through a distal tip opening 156. In addition, the instrument 140 is equipped with a light source 164 such as an arc lamp which emits light having at least one predetermined wavelength, the light being directed to the distal tip opening 156 via optical fibers 176, the light being reflected and returned via optical fibers 172 or other known means to a spectrometer 168. The spectra of such emissions are examined at certain specified emission wavelengths with regard to a reference standard. This technique has been demonstrated to be effective for determining the presence of dental caries as well as cancer diagnosis for specified anatomical targets, such as the cervix. By measuring the wavelength of light emitted by the medical target 20, for example, a determination can be made using fluorescence as to the presence of otitis media.

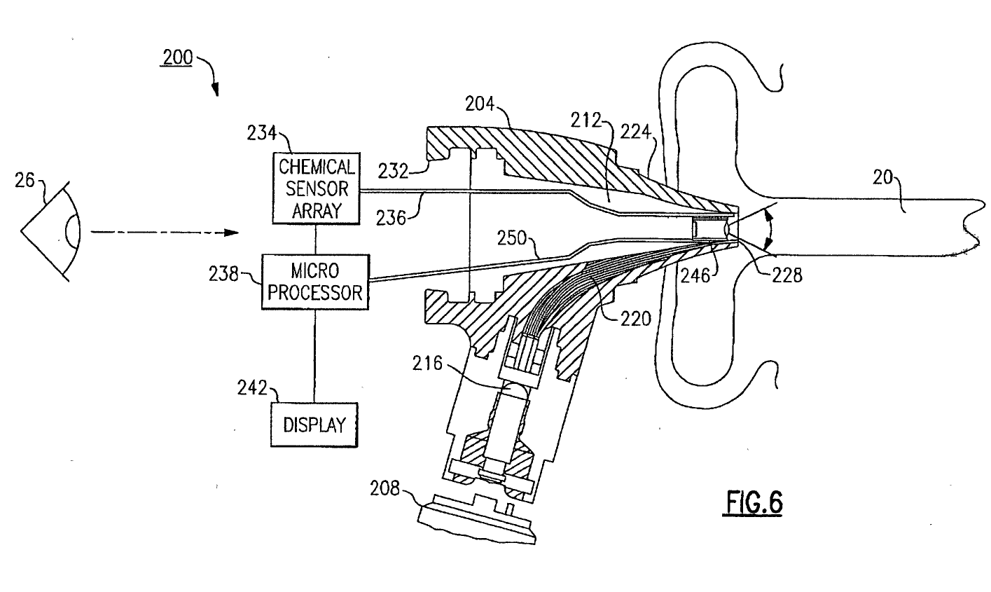

According to another pathogen detection method, and referring to FIG. 6, an otoscope 200 in accordance with another embodiment of the invention includes an instrument head 204 which is attached to the top of a handle 208. At least one electronic chemical sensing element is disposed within the instrument head. According to the present embodiment, an array 234 of chemical sensing elements are disposed on a substrate (not shown) within the instrument head 204 in the vicinity of the distal tip opening 228. The array of sensors can be attached e.g., by epoxy, frit, adhesive, or other conventional means to the substrate. Alternately, the sensor array 234 can be provided as part of a plug-in electrical module (not shown) having connectors which mount to a mating portion of the substrate. The chemical sensor array 234 according to this embodiment are miniature polymer gas sensors such as those described in U.S. Pat. No. 5,571,401 to Lewis et al., U.S. Pat. No. 5,882,497 to Persaud et al., U.S. Pat. No. 6,033,601 to Persaud et al., U.S. Pat. No. 6,093,308 to Lewis, and U.S. Pat. No. 6,013,229 to Lewis, the entire contents of each herein being incorporated by reference. It will be apparent, however, that other known chemical sensing elements such as organic gas sensors, conductive composites, metal oxide sensors, chemically reactive sensors, such as dye sensors or chromatic sensors, metal oxide field effect transistors, surface acoustic wave (SAW) sensors, piezoelectric sensors, and others, can also be substituted. Each of the sensing elements of the array 234 are capable of detecting the presence of trace amounts of a specific fluid (e.g., a liquid, gas, or vapor), the presence of which causes a parametric change. This parametric change can be electrical such as resistance, capacitance, conductance, transconductance, impedance, voltage, resonant frequency, or other perceived electrical or optical or chemical change. Each of the chemical sensing elements are selected and attuned as described in the above cross-referenced patents, to react differently to various fluids emitted, thereby creating a signature(s) for each bacteria or virus which is sensed thereby. A preferred embodiment of a chemical sensing element array suitable for use in the herein described embodiment is described in greater detail in U.S. Ser. No. 09/663,698 (Attorney Docket 281_{—}309 sub (2)), the entire contents being herein incorporated herein by reference.

The chemical sensing element array 234 can be presented in the distal insertion portion 224 of the instrument 200 or alternately, a sample of the environment can be directed through a tube 236 to the array 234 which can be disposed in the proximal end 232 or other location of the instrument head 204 or handle 208. The array 234 could also be located remotely and tether to the instrument 200.

A series of electrical traces (not shown) extend from the separate sensing elements of the array 234 along the supporting substrate (not shown) and further extend to a microprocessor 238. The microprocessor 238 includes certain integrated processing electronics including an analog to digital converter as well as timing and control circuitry which is used in conjunction with a reference crystal (not shown). Each are provided in order to detect the amount of parametric change by each of the sensors of the array 234 for processing. The microprocessor 238 also includes sufficient memory for storing the values of the signals from each of the sensing elements and can further include additional memory for further processing such as inclusion of a lookup table for comparing the values of the stored signals in order to determine the presence of a bacteria, virus, or other pathogen. The microprocessor 238 is further connected to a display 242, such as an LED, which indicates to the care giver when a particular condition is detected.

Alternately, the device can include an antenna (not shown) which is electrically connected to the microprocessor 238 to permit wireless RF or IR transmission to a remote processor (not shown) having additional processing capability.

As shown in FIG. 6, the chemical sensor array 234 can be utilized/disposed to permit visual otoscopy through the proximal end 232 by the eye 26 or/and a thermal sensing or video imaging assembly 246 can be positioned at the distal tip opening 228 of the instrument and interconnected electrically to the microprocessor 238 through transmission line 250. Illumination is provided by light source 216 disposed in the neck of the instrument 200 to a bundle of optical fibers 220 disposed in relation to the distal tip opening 228.

It should be further noted that the herein described instruments can obtain a video image using the device of FIG. 2 and superimpose an image, such as obtained using the spectroscopic device of FIG. 5 and/or the thermal sensing array of FIG. 3. Each of the above can be used with the display 76 such as that shown in FIG. 2 to enhance or further improve the diagnosis of a patient.

Parts List for FIGS. 1-6

10 otoscopic diagnostic instrument

14 handle

19 distal insertion portion

20 ear canal

23 proximal portion

24 instrument head

25 eyepiece

26 eye

27 distal tip opening

28 optical axis

29 optical fiber bundle

31 light source

35 insufflation means

40 otoscope

42 neck

43 light source

44 instrument head

45 video imaging assembly

48 distal insertion portion

50 distal tip opening

52 electronic imager

54 optical axis

56 lens cell

58 fiberoptic bundle

60 microphone

62 line

64 speaker

66 line

68 pump

70 line

72 transmission lines

76 video display

100 otoscopic diagnostic instrument

104 instrument head

106 distal insertion portion

107 handle

108 light source

109 optical axis

112 optical fiber bundle

114 distal tip opening

116 thermal sensing array

120 proximal end

124 housing

126 focusing lens system

140 diagnostic instrument

144 instrument head

146 handle

148 interior

152 distal insertion portion

156 distal tip opening

160 proximal end

164 light source

168 spectrometer

172 optical fibers

176 optical fibers

200 instrument

204 instrument head

208 handle

212 interior

216 light source

220 optical fiber bundle

224 distal insertion portion

228 distal tip opening

232 proximal end

234 chemical sensor array

236 tube

238 microprocessor

242 display

246 imaging assembly

It should be readily apparent that certain modifications and variations are possible within the ambits of the herein described invention which are embodied in accordance with the following claims.

We claim:

1. A medical diagnostic instrument comprising:
at least one chemical sensor for determining the presence of a fluid indicative of a disease; and
viewing means for viewing a medical target of interest.

2. An instrument as recited in claim 1, including an insertion portion capable of being inserted to a predetermined distance into the ear canal of a patient, wherein said at least one chemical sensor is disposed within said insertion portion.

3. An instrument as recited in claim 1, wherein said viewing means includes an eyepiece aligned with an insertion portion along an optical axis.

4. An instrument as recited in claim 1, wherein said viewing means includes a image sensor.

5. An instrument as recited in claim 4, wherein said image sensor is disposed in said insertion portion of said instrument.

6. An instrument as recited in claim 1, including temperature measuring means for determining the body temperature of a patient.

7. An instrument as recited in claim 6, wherein said temperature measuring means includes at least one IR sensor capable of providing a thermal image of the medical target.

8. An instrument as recited in claim 6, wherein said temperature measuring means includes means for determining the pulse rate of the patient.

9. An instrument as recited in claim 1, including fluid detecting means for detecting the presence of fluid in the middle ear.

10. An instrument as recited in claim 1, including pressure measuring means for measuring pressure variations in the middle ear.

11. An instrument as recited in claim 10, wherein said pressure measuring means includes at least one of at least one of pneumatic otoscopy, oto-reflectance, impedance reflectance, and tympanometry means for determining pressure variations in the middle ear.

12. An instrument as recited in claim 9, wherein said fluid detecting means includes at least one of pneumatic otoscopy, oto-reflectance, acoustic reflectance, impedance reflectance, and tympanometry means for detecting the presence of fluid in the middle ear.

13. An instrument as recited in claim 1, including a microphone capable of determining the respiration rate of a patient.

14. An instrument as recited in claim 1, including spectroscopic means for determining the presence of pathogens, said spectroscopic means including a light source capable of emitting predetermined wavelengths of light and detecting means capable of detecting predetermined wavelengths of light emitted from the medical target.

15. An instrument as recited in claim 2, including means for determining hearing in the inner ear.

16. An instrument as recited in claim 15, wherein said hearing determining means includes at least one of otoreflectance means and otoacoustic emission means for determining hearing in the inner ear.

17. An instrument as recited in claim 1, wherein said instrument is capable of inserting said at least one chemical sensor into an anatomical body cavity.

18. An instrument as recited in claim 17, wherein said anatomical body cavity is the throat.

19. An instrument as recited in claim 17, wherein said anatomical body cavity is the ear.

20. An instrument as recited in claim 17, wherein said anatomical body cavity is the nose.

21. An otological instrument comprising:
oto-reflectance means for determining at least one of detecting a middle-ear disorder and hearing in the inner ear; and
pathogen sensing means for determining the presence of a disease.

22. An instrument as recited in claim 21, including viewing means for viewing the interior of said ear.

23. An instrument as recited in claim 22, including an insertion portion, wherein said viewing means includes an eyepiece aligned with said insertion portion.

24. An instrument as recited in claim 22, wherein said viewing means includes a image sensor.

25. An instrument as recited in claim 24, wherein said image sensor is disposed in an insertion portion of said instrument.

26. An instrument as recited in claim 21, including temperature measuring means for determining the body temperature of the patient.

27. An instrument as recited in claim 26, wherein said temperature measuring means includes at least one IR sensor capable of providing a thermal image of the interior of said ear.

28. An instrument as recited in claim 26, wherein said temperature measuring means includes means for determining the pulse rate of the patient.

29. An instrument as recited in claim 21, including pneumatic otoscopy means for stimulating the tympanic membrane.

30. An instrument as recited in claim 21, including a microphone disposed in an insertion portion, said microphone being capable of determining the respiration rate of a patient.

31. An instrument as recited in claim 21, wherein said pathogen sensing means includes spectroscopic means for detecting the presence of pathogens including a light source capable of emitting predetermined wavelengths of light and detecting means capable of detecting predetermined wavelengths of light at least one of emitted or reflected within the ear.

32. An instrument as recited in claim 21, wherein said pathogen sensing means includes at least one chemical sensor for determining the presence of a fluid indicative of a disease.

33. A medical diagnostic instrument comprising:
viewing means for viewing a medical target; and
temperature measuring means for measuring body temperature of a patient, said temperature measuring means including at least one sensor capable of producing an output signal which is indicative of temperature and processing electronics for processing output signals from said at least one sensor.

34. An instrument as recited in claim 33, wherein said medical target is an anatomical body cavity.

35. An instrument as recited in claim 34, wherein said body cavity is the throat.

36. An instrument as recited in claim 34, wherein said body cavity is the ear.

37. An instrument as recited in claim 34, wherein said body cavity is the nose.

38. The instrument as recited in claim 36, including means for measuring pressure variations within the middle ear.

39. The instrument as recited in claim 38, wherein said pressure measuring means includes at least one of pneumatic otoscopy, oto reflectance, impedance reflectance, and tympanometry means for determining pressure variations in the middle ear.

40. The instrument as recited in claim 36, including means for determining hearing in the inner ear.

41. The instrument as recited in claim 40, wherein said hearing determining means includes at least one otoreflectance means and otoacoustic emission means for determining hearing in the middle ear.

42. The instrument as recited in claim 33, including an insertion portion and an optical system aligned with said insertion portion including an eyepiece which receives a focused image of a target from said optical system.

43. The instrument as recited in claim 42, wherein said viewing means includes an optical system aligned with said insertion portion and an image sensor, said image sensor and said optical system being aligned along an optical axis.

44. The instrument as recited in claim 43, wherein said image sensor is disposed within insertion portion and aligned with focusing optics for guiding a focused optical image viewed through said insertion portion.

45. The instrument as recited in claim 43, including a display for displaying a video image as captured by said image sensor, said display being provided on an eyepiece portion of said instrument.

46. The instrument as recited in claim 33, including sensing means for detecting the presence of pathogens in the medical target.

47. The instrument as recited in claim 46, wherein said pathogen sensing means includes spectroscopic means including light emitting means for emitting a light having a predetermined wavelength and means for analyzing a spectrum of light which is at least one of reflected and fluoresced from the interior of said medical target.

48. The instrument as recited in claim 46, wherein said pathogen sensing means includes at least one chemical sensor capable of detecting the presence of a disease and emitting an output signal indicative of the relative amount thereof.

49. An instrument as recited in claim 33, wherein said temperature measuring means includes means for determining pulse.

50. An instrument as recited in claim 33, including a microphone capable of determining the respiration rate of a patient.

51. The instrument as recited in claim 36, including means for detecting the presence of fluid in the middle ear.

52. The instrument as recited in claim 51, wherein said fluid detecting means includes at least one of pneumatic otoscopy, acoustic reflectance, oto-reflectance, impedance reflectance, and tympanometry means for detecting the presence of fluid in the middle ear.

53. An instrument for determining the condition of an ear, said instrument comprising:
means for viewing the ear interior; and
means for measuring pressure variations within the ear.

54. The instrument as recited in claim 53, including temperature measuring means for measuring body temperature of a patient, said temperature measuring means including at least one infrared sensor capable of producing an output signal which is indicative of temperature.

55. The instrument as recited in claim 54, including processing electronics for processing output signals from said at least one infrared sensor.

56. The instrument as recited in claim 54, wherein said temperature measuring means includes means for determining pulse rate.

57. The instrument as recited in claim 53, wherein said pressure measuring means includes at least one of pneumatic otoscopy, impedance reflectance, oto-reflectance, and tympanometry means for measuring pressure variations in the middle ear.

58. The instrument as recited in claim 53, including means for determining hearing in the inner ear.

59. The instrument as recited in claim 58, wherein said hearing determining means includes at least one of otoreflectance and otoacoustic emission means for determining hearing in the inner ear.

60. The instrument as recited in claim 53, including an insertion portion, said viewing means including an optical system aligned with said insertion portion and an eyepiece which receives a focused image of a target from said optical system.

61. The instrument as recited in claim 60, wherein said viewing means includes an optical system aligned with said insertion portion and an imaging sensor, said imaging sensor and said optical system being aligned along an optical axis.

62. The instrument as recited in claim 61, wherein said imaging sensor is disposed within insertion portion and aligned with focusing optics for guiding a focused optical image viewed through said insertion portion.

63. The instrument as recited in claim 61, including a display for displaying a video image as captured by said imaging sensor, said display being provided on an eyepiece portion of said instrument.

64. The instrument as recited in claim 53, including sensing means for detecting the presence of pathogen in the ear.

65. The instrument as recited in claim 64, wherein said sensing means includes spectroscopic means includes light emitting means for emitting a light having at least one predetermined wavelength and means for analyzing a spectrum of light which is at least one of reflected and fluoresced from the interior of said ear.

66. The instrument as recited in claim 64, wherein said sensing means includes at least one chemical sensor capable of detecting th e presence of a disease and emitting an output signal indicative of the relative amount thereof.

67. The instrument as recited in claim 53, including a microphone insertable in the ear capable of detecting the respiration rate of a patient.

68. A diagnostic instrument comprising:
means for viewing a medical target; and
sensing means for detecting the presence of pathogen at the medical target.

69. The instrument as recited in claim 68, wherein said medical target is an anatomical body cavity.

70. The instrument as recited in claim 69, wherein said body cavity is the throat.

71. The instrument as recited in claim 69, wherein said body cavity is the ear.

72. The instrument as recited in claim 69, wherein said body cavity is the nose.

73. The instrument as recited in claim 68, including temperature measuring means for measuring the body temperature of a patient, said temperature measuring means including at least one infrared sensor capable of producing an output signal which is indicative of temperature.

74. The instrument as recited in claim 73, wherein said pathogen detecting means includes said temperature measuring means.

75. The instrument as recited in claim 73, including processing electronics for processing output signals from said at least one infrared sensor and said sensing means.

76. The instrument as recited in claim 73, wherein said temperature measuring means includes means for determining pulse rate.

77. The instrument as recited in claim 71, including pressure measuring means for measuring pressure variations within the middle ear.

78. The instrument as recited in claim 77, wherein said pressure measuring means includes at least one of pneumatic otoscopy, otoreflectance impedance reflectance and tympanometry means for measuring pressure variations in the middle ear.

79. The instrument as recited in claim 71, including fluid detecting means including at least one of pneumatic otoscopy, acoustic reflectance, impedance reflectance, oto reflectance and tympanometry means for determining pressure variations in the middle ear.

80. The instrument as recited in claim 71, including means for determining hearing in the inner ear.

81. The instrument as recited in claim 80, wherein said hearing determining means includes at least one of otoreflectance and otoacoustic emission means for determining hearing in the inner ear.

82. The instrument as recited in claim 68, including an insertion portion, said viewing means including an optical system aligned with said insertion portion and an eyepiece which receives a focused image of a target from said optical system.

83. The instrument as recited in claim 82, wherein said viewing means includes an optical system aligned with said insertion portion and an imaging sensor, said imaging sensor and said optical system being aligned along an optical axis.

84. The instrument as recited in claim 83, wherein said imaging sensor is disposed within insertion portion and aligned with focusing optics for guiding a focused optical image viewed through said insertion portion.

85. The instrument as recited in claim 83, including a display for displaying a video image as captured by said imaging sensor, said display being provided on an eyepiece portion of said instrument.

86. The instrument as recited in claim 68, wherein said pathogen sensing means includes spectroscopic means includes light emitting means for emitting a light having at least one predetermined wavelength and means for analyzing a spectrum of light which is at least one of reflected and fluoresced from the interior of said medical target.

87. The instrument as recited in claim 68, wherein said pathogen sensing means includes at least one chemical sensor capable of detecting the presence of a disease and emitting an output signal indicative thereof.

88. An instrument as recited in claim 68, including a microphone positionable in relation to the medical target to determine the respiration rate of a patient.

89. An instrument for determining conditions of the ear, said instrument comprising:
temperature measuring means for determining the body temperature of a patient, said temperature measuring means including at least one miniature infrared sensor capable of providing a thermal image of the interior of the ear; and
pressure measuring means for measuring pressure variations within the middle ear.

90. The instrument as recited in claim 89, wherein said pressure measuring means includes at least one of pneumatic otoscopy, otoreflectance, impedance reflectance, and tympanometry means for measuring pressure variations in the middle ear.

91. The instrument as recited in claim 89, wherein said temperature measuring means includes means of measuring the pulse based on transient changes in the thermal image produced by said infrared sensor array.

92. The instrument as recited in claim 89, including a microphone arranged so as to measure the respiratory rate of said patient.

93. The instrument as recited in claim 89, including means for determining hearing, said hearing determining means including at least one of otoreflectance and otoacoustic emission means for determining hearing in the inner ear.

94. The instrument as recited in claim 89, including fluid detecting means including at least one of pneumatic otoscopy, acoustic reflectance, impedance reflectance, oto reflectance, and tympanometry means for detecting fluid in the middle ear.

95. The instrument as recited in claim 89, including sensing means for sensing the presence of bacteria and viruses indicative of otitis.

96. The instrument as recited in claim 95, wherein said sensing means includes spectroscopic means including light emitting means for emitting a light having at least one predetermined wavelength and means for analyzing a spectrum of light which is at least one of reflected and fluoresced from the interior of said ear.

97. The instrument as recited in claim 95, wherein said sensing means includes at least one chemical sensor capable of detecting the presence of a disease and emitting an output signal indicative thereof.

98. The instrument as recited in claim 89, including viewing means for viewing the interior of the ear, said temperature measuring means and said pressure measuring means being disposed in relation to said viewing means so as not to interfere therewith.

99. The instrument as recited in claim 98, wherein said viewing means includes an image sensor.

100. The instrument as recited in claim 99, wherein said image sensor is disposed in an insertion portion of said instrument.

101. The instrument as recited in claim 99, wherein said viewing means includes an eyepiece aligned with an insertion portion.

102. A medical diagnostic instrument comprising:
temperature measuring means for determining the body temperature of a patient, said temperature measuring means including an array of infrared sensors capable of providing a thermal image of a medical target; and
sensing means for sensing the presence of pathogens indicative of an anomalous medical condition.

103. The instrument as recited in claim 102, wherein said medical target is an anatomical body cavity.

104. The instrument as recited in claim 103, wherein said body cavity is the throat.

105. The instrument as recited in claim 103, wherein said body cavity is the ear.

106. The instrument as recited in claim 103, wherein said body cavity is the nose.

107. The instrument as recited in claim 102, wherein said pathogen sensing means includes spectroscopic means including light emitting means for emitting a light having a predetermined wavelength and means for analyzing a spectrum of light which is at least one of reflected and fluoresced from the interior of said medical target.

108. The instrument as recited in claim 102, wherein said pathogen sensing means includes at least one chemical sensor capable of detecting the presence of a disease and emitting an output signal indicative thereof.

109. The instrument as recited in claim 105, including pressure measuring means for measuring pressure variations in the ear.

110. The instrument as recited in claim 109, wherein said pressure measuring means includes at least one of pneumatic otoscopy, otoreflectance, impedance reflectance, and tympanometry means for measuring pressure variations in the middle ear.

111. The instrument as recited in claim 105, including means for determining hearing in the inner ear.

112. The instrument as recited in claim 102, wherein said temperature measuring means includes means of measuring pulse rate based on transient changes in the thermal image produced by said infrared sensor array.

113. The instrument as recited in claim 102, including a microphone arranged so as to measure the respiratory rate of said patient.

114. The instrument as recited in claim 102, including viewing means for viewing said medical target, said temperature measuring means and said sensing means being disposed in relation to said viewing means so as not to interfere therewith.

115. The instrument as recited in claim 114, wherein said viewing means includes an image sensor.

116. The instrument as recited in claim 115, wherein said image sensor is disposed in an insertion portion of said instrument.

117. The instrument as recited in claim 114, wherein said viewing means includes an eyepiece aligned with an insertion portion.

118. The instrument as recited in claim 105, including means for detecting fluid in the middle ear.

119. The instrument as recited in claim 118, wherein said fluid detecting means includes at least one of pneumatic otoscopy, acoustic reflectance, otoreflectance, impedance reflectance, and tympanometry means for detecting fluid in the middle ear.

120. A medical instrument system capable of comprehensively examining the ear, said system comprising:
a housing having an insertion portion sized for fitting a predetermined distance within the ear canal of a patient;
means for examining the outer ear;
means for examining the middle ear; and
means for examining the inner ear of a patient, each of said examining means being at least partially contained within said housing.

121. The system as recited in claim 120, including means for determining certain vital signs of the patient.

122. The system as recited in claim 121, wherein said vital signs determining means includes temperature measuring means for determining the body temperature of a patient.

123. The system as recited in claim 122, wherein said temperature measuring means includes an array of sensors capable of providing a thermal image of the ear interior including the tympanic membrane and processing electronics for processing output signals produced by said array.

124. The system as recited in claim 123, wherein the processing electronics of said temperature measuring means includes means for determining transient changes in the thermal image produced by said array so as to determine pulse rate.

125. The system as recited in claim 121, wherein said vital signs determining means includes a microphone for detecting inspiration and expiration sounds of a patient and processing means for distinguishing between inspiration and respiratory sounds in order to determine respiratory rate thereof.

126. The system as recited in claim 120, wherein at least one of said inner ear, said outer ear and said middle ear examining means includes sensing means for sensing the presence of bacteria and viral conditions.

127. The system as recited in claim 126, wherein said bacteria and viral condition sensing means includes spectroscopic means having light emitting means for emitting a light having at least one predetermined wavelength and means for analyzing a spectrum of light which is at least one of reflected and fluoresced from the interior of said ear.

128. The system as recited in claim 126, wherein said sensing means includes at least one chemical sensor capable of emitting an output signal indicative of the presence of a disease and processing means for processing said signal.

129. The system as recited in claim 128, wherein said at least one chemical sensor is disposed within an insertion portion of said instrument.

130. The system as recited in claim 128, wherein said at least one chemical sensor is disposed within a handle portion of said instrument.

131. An otological instrument comprising:
pressure determining means for determining pressure variations in the middle ear; and
means for sensing the presence of pathogens in the ear.

132. The instrument as recited in claim 131, including viewing means for viewing the interior of the ear.

133. The instrument as recited in claim 131, including an insertion portion for inserting a predetermined distance into the ear canal.

134. The instrument as recited in claim 132, wherein said viewing means includes an image sensor.

135. The instrument as recited in claim 133, including an eyepiece attached to said insertion portion.

136. The instrument as recited in claim 131, wherein said pathogen sensing means includes spectroscopic means including light emitting means for emitting a light having at least one predetermined wavelength and means for analyzing a spectrum of light which is at least one of reflected and fluoresced from the interior of an ear.

137. The instrument as recited in claim 131, wherein said pathogen sensing means includes at least one chemical sensor capable of detecting the presence of a disease and emitting an output signal indicative thereof.

138. The instrument as recited in claim 131, including means for determining hearing in the inner ear.

139. The instrument as recited in claim 138, wherein said hearing determining means includes at least one of oto-reflectance means and otoacoustic emission means for determining hearing in the inner ear.

140. The instrument as recited in claim 131, wherein said pressure determining means includes at least one of pneumatic otoscopy, impedance reflectance, otoreflectance and tympanometry means for determining pressure variations in the middle ear.

141. The instrument as recited in claim 131, including fluid detecting means for detecting the presence of fluid in the middle ear.

142. The instrument as recited in claim 131, wherein said fluid detecting means includes at least one of pneumatic otoscopy, acoustic reflectance, oto-reflectance, impedance reflectance, and tympanometry means for detecting the presence of fluid in the middle ear.

143. The instrument as recited in claim 131, including temperature measuring means for determining the body temperature of a patient.

144. The instrument as recited in claim 143, wherein said temperature measuring means includes an array of sensors capable of providing a thermal image of the tympanic membrane.

145. The instrument as recited in claim 143, wherein said temperature measuring means includes means for determining the pulse rate of a patient.

146. The instrument as recited in claim 131, including a microphone capable of determining the respiration rate of a patient.

147. A method for examining a medical target using a combination otoscope, said method comprising the steps of:
disposing at least one chemical sensing element in an otoscope, said at least one chemical sensing element being capable of detecting at least one fluid indicative of a disease;
performing an otological examination of an ear of patient;
placing said at least one chemical sensing element into the ear; and
indicating when said at least one fluid is detected.

148. A method as recited in claim 147, including the steps of:
placing said at least one chemical sensing element into another body cavity of said patient; and
indicating when at least one fluid is detected.

149. An instrument as recited in claim 7, wherein said viewing means includes an image sensor, said instrument further including display means for displaying at least one of a thermal image and at least one of a continuous and still video image of said medical target.

150. An instrument as recited in claim 149, including spectroscopic means for determining the presence of pathogens, said spectroscopic means including a light source capable of emitting predetermined wavelengths of light and detecting means capable of detecting predetermined wavelengths of light emitted within the medical target said display means being capable of displaying at least one of a thermal image, a video image, and a spectroscopic image of said medical target.

151. An instrument as recited in claim 27, including viewing means for viewing the interior of said ear including an image sensor, said instrument further including display means for displaying at least one of a video image and a thermal image of the interior of the ear.

152. An instrument as recited in claim 151, including spectroscopic means for determining the presence of pathogens, said spectroscopic means including a light source capable of emitting predetermined wave lengths of light and detecting means capable of detecting predetermined wave lengths of light emitted within the medical target said display means being capable of displaying at least one of a thermal image, a video image, and a spectroscopic image.

153. An instrument as recited in claim 45, wherein said display is capable of displaying at least one of a video image and a thermal image as captured by said temperature measuring means.

154. An instrument as recited in claim 153, including spectroscopic means for determining the presence of pathogens, said spectroscopic means including a light source capable of emitting predetermined wave lengths of light and detecting means capable of detecting predetermined wave lengths of light emitted within the medical target said display means being capable of displaying at least one of a thermal image, a video image, and a spectroscopic image of said medical target.

155. An instrument as recited in claim 55, wherein said viewing means includes an image sensor and said instrument further including display means connected to said processing electronics and said image sensor for producing at least one of a video image and a thermal image of the ear interior.

156. An instrument as recited in claim 155, including spectroscopic means for determining the presence of pathogens, said spectroscopic means including a light source capable of emitting predetermined wave lengths of light and detecting means capable of detecting predetermined wave lengths of light emitted within the medical target said display means being capable of displaying at least one of a thermal image, a video image, and a spectroscopic image.

157. An instrument as recited in claim 75, including viewing means for viewing the ear interior including an image sensor, said instrument further including display means connected to said processing electronics and said image sensor for producing at least one of a video image and a thermal image of the ear interior.

158. An instrument as recited in claim 157, including spectroscopic means for determining the presence of pathogens, said spectroscopic means including a light source capable of emitting predetermined wave lengths of light and detecting means capable of detecting predetermined wave lengths of light emitted within the medical target said display means being capable of displaying at least one of a thermal image, a video image, and a spectroscopic image.

159. An instrument as recited in claim 99, including display means connected to said image sensor and said temperature measuring means for displaying at least one of a video image and a thermal image of the ear.

160. An instrument as recited in claim 159, including spectroscopic means for determining the presence of pathogens, said spectroscopic means including a light source capable of emitting predetermined wave lengths of light and detecting means capable of detecting predetermined wave lengths of light emitted within the medical target said display means being capable of displaying at least one of a thermal image, a video image, and a spectroscopic image.

161. An instrument as recited in claim 116, including display means connected to said image sensor and said temperature measuring means for displaying at least one of a video image and a thermal image of the ear.

162. An instrument as recited in claim 161, including spectroscopic means for determining the presence of pathogens, said spectroscopic means including a light source capable of emitting predetermined wave lengths of light and detecting means capable of detecting predetermined wave lengths of light emitted within the medical target said display means being capable of displaying at least one of a thermal image, a video image, and a spectroscopic image.

163. A system as recited in claim 123, including viewing means including an image sensor for viewing the ear interior, and display means for displaying at least one of a video image and a thermal image of the ear.

164. A system as recited in claim 163, including spectroscopic means for determining the presence of pathogens, said spectroscopic means including a light source capable of emitting predetermined wave lengths of light and detecting means capable of detecting predetermined wave lengths of light emitted within the medical target said display means being capable of displaying at least one of a thermal image, a video image, and a spectroscopic image.

165. A method as recited in claim 148, wherein said combination otoscope includes video imaging means for producing a video image of said ear and body cavity and thermal imaging means for producing a thermal image thereof, said method including the additional step of displaying at least one of a thermal image and a video image.

166. An instrument as recited in claim 144, including viewing means for viewing the interior of the ear, said viewing means including at least one image sensor and a display, said display being connected to said image sensor and said temperature measuring means for displaying at least one of a video image and a thermal image.
